(19) United States
(12) Patent Application Publication
(10) Pub. No.: US 2004/0009126 A1
(43) Pub. Date: Jan. 15, 2004

(54) Inhalation system for prevention and treatment of intracellular infections

(75) Inventors: Frank G. Pilkiewicz, Princeton Junction, NJ (US); Lawrence T. Boni, Monmouth Junction, NJ (US); Constance Mackinson, Skillman, NJ (US); Joel B. Portnoff, Langhorne, PA (US); Anthony Scotto, Washington Crossing, PA (US); Fangjun Wu, Livingston, NJ (US); Brian Miller, Mercerville, NJ (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Transave, Inc., Monmouth Junction, NJ (US)

(21) Appl. No.: 10/383,173

(22) Filed: Mar. 5, 2003

Related U.S. Application Data
(60) Provisional application No. 60/361,809, filed on Mar. 5, 2002.

(51) Int. Cl. A61L 9/04; A61K 9/14

(52) U.S. Cl. 424/46

(57) ABSTRACT

An inhalation system comprising an antiinfective agent in particle form, the antiinfective agent being directed toward prevention and treatment of intracellular infection, and an inhalation device, and a method of use of the system.

The present application claims the benefit of the priority of U.S. Provisional Patent Application No. 60/361,809 filed Mar. 5, 2002, the disclosure of which is hereby incorporated by reference as if fully set forth herein.

The present invention relates to a system for administering antiinfective agents by inhalation. More particularly the present invention relates to the treatment of pulmonary infections by administering antibacterial agents or antiviral agents by inhalation.

The lungs act as a portal to the body by means of uptake of materials by cells of the lung, such as alveolar macrophages. As a result antiinfective agents, such as antibacterial agents and antiviral agents, can be administered through the lung portal. Such systematic treatment can avoid hepatic first pass inactivation and allow for lower doses with fewer side effects.

Inhalation can specifically be used to treat pulmonary infections, and more particularly intracellular infections that involve uptake, persistence and transport of the bacteria by the pulmonary macrophages of the lungs. Such bacteria include, Bacillus anthracis, Listeria monocytogenes, Staphylococcus aureus, Salmenellolosis, Pseudomonas aeruginosa, Yersina pestis, Mycobacterium leprae, M. africanum, M. asiaticum, M. avium-intracellulare, M. chelonei subsp. abscessus, M. fallax, M. fortuitum, M. kansasii, M. leprae, M. malmoense, M. shimoidei, M. simiae, M. szulgai, M. xenopi, M. tuberculosis, Brucella melitensis, Brucella suis, Brucella abortus, Brucella canis, Legionella pneumonophilia, Francisella tularensis, pneumocystis carinii and other microorganisms that are intracellular and can involve uptake and transport by the lungs' macrophages in disseminating the bacterial infection.

The administration of an antiinfective agent for treatment of infection by inhalation is particularly attractive for several reasons. Firstly, inhalation is a more localized administration of the antiinfective agent and can therefore be more effective in terms of timing and ratio of antiinfective agent reaching the infection. Further, inhalation can be easier to use. In some instances the antiinfective agent can even be self-administered by inhalation, which tends to improve patient compliance and reduce costs.

Although inhalation of antiinfective agents appears to be an attractive alternative to injection for treating intracellular infection, use of conventional inhalation systems has been slowed by several significant disadvantages: (1) due to the physiology of the lung, antiinfective agents that are administered by inhalation quickly clear the lung and, therefore, yield short term therapeutic effects. This rapid clearance can result in the antiinfective agent having to be administered more frequently and, therefore, adversely affecting patient compliance and increasing the risk of side effects; (2) conventional inhalation systems do not enhance the targeted delivery of antiinfective agents to the site of disease; (3) inhalation formulations are susceptible to both chemical and enzymatic in-vivo degradation. This degradation is particularly detrimental to peptide and protein formulations; and (4) due to aggregation and lack of stability, formulations of high molecular weight compounds like peptides and proteins are not effectively administered as aerosols, nebulized sprays or as dry powder formulations.

The present invention can overcome these disadvantages in treatment of infection by inhalation, and offers new advantages to inhalation that can enhance the therapeutic index of a currently used antiinfective agent. The invention can be used for the successful entrapment and delivery of both low and high molecular weight compounds. The present invention provides for particulate bioactive agents, such as lipid particles, which can be administered by inhalation as part of a delivery system.

### SUMMARY OF THE INVENTION

A system for delivery of an antiinfective agent comprising a pharmaceutical fomulation comprising a particle comprising an antiinfective agent directed to prevention and treatment of intracellular infections caused by an infective, the pharmaceutical formulation comprising particles with a diameter of between approximately 0.01 microns and approximately 2.0 microns and an inhalation delivery device.

The pharmaceutical formulation of the antiinfective agent is, in preferred forms, a particle of the antiinfective agent, a particle made up of the antiinfective agent and one or more pharmaceutically acceptable excipients, a non-covalent modification of the antiinfective agent, a mixture of the antiinfective agent and a lipid, the antiinfective agent and a mixture of phospholipids, a lipid complex, a lipid clathrate, a proliposome, a liposome, or a polymer formulation of the antiinfective agent.

The particles administered by inhalation can be selectively taken up by the pulmonary macrophages, the lymphatics and the organs that also contain the intracellular infection so that the particles are effective in treating pulmonary infections, particularly intracellular infections. The particles can also be administered prophylactically when the threat of contracting a pulmonary infection, particularly an intracellular infection, exists.

The present invention includes a method wherein the system is employed for the prevention and treatment of a medical condition.

The present invention covers a system for delivery of an antiinfective agent comprising a pharmaceutical formulation comprising a particle of an antiinfective agent directed to prevention and treatment of intracellular infections in the lung caused by an infective agent, the pharmaceutical formulation comprising particles with a diameter of between approximately 0.01 microns and approximately 2.0 microns and, an inhalation delivery device. Particles can have a diameter of between approximately 0.01 microns and approximately 1.0 micron. Particles can have a diameter of between approximately 0.01 microns and approximately 0.5 microns. Particles can have a diameter of between of between approximately 0.02 microns and approximately 0.5 microns.

The infective agent included in the scope of the present invention can be a bacteria. The bacetria can be selected from Bacillus anthracis, Listeria monocytogenes, Staphylococcus aureus, Salmenellolosis, Pseudomonas aeruginosa, Yersina pestis, Mycobacterium leprae, M. africanum, M. asiaticum, M. avium-intracellulare, M. chelonei subsp. abscessus, M. fallax, M. fortuitum, M. kansasii, M. leprae, M. malmoense, M. shimoidei, M. simiae, M. szulgai, M. xenopi, M. tuberculosis, Brucella melitensis, Brucella suis, Brucella abortus, Brucella canis, Legionella pneumonophilia, Francisella tularensi, pneumocystis carinii and mycoplasma.

The infective agent included in the scope of the present invention can be a virus. The virus can be one of hantavirus, respiratory syncytial virus, influenza, and viral pneumonia.

The pharmaceutical formulation of a particle comprising the antiinfective agent can be in particle form, can comprise a mixture of the antiinfective agent and one or more excipients, can comprise a non-covalent modification of the antiinfective agent such as a salt, for example the sodium, potassium, lithium, sulfate, citrate, phosphate, calcium, magnesium or iron salt of the antiinfective agent, can comprise the antiinfective agent and the one or more lipids being formulated as a lipid mixture, can comprise a mixture of phospholipids including one or more phospholipids selected from the group consisting of phosphatidylcholines, phosphatidylglycerols, phosphatidylserines, phosphotidylinositols, phosphatidylethanolamines, sphingomyelins, ceramides, and steroids, can comprise the antiinfective agent and a lipid, the antiinfective agent and the lipid being formulated as a lipid complex and can comprise a liposome. The liposome can comprise a multilamellar vesicle, a small unilamellar vesicle or other liposomes

The antiinfective agent to lipid ratio is preferably from 10:1 to 1:1000 by weight.

The pharmaceutical formulation can further comprise a mixture of one or more steroids.

The present invention also includes a method for treatment of intracellular infection in its scope, the method comprising:

a) providing a pharmaceutical fomulation comprising a particle comprising an ant linfective agent, the antiinfective agent being directed to treatment of intracellular infections in the lung, the pharmaceutical formulation comprising particles with a diameter of between approximately 0.01 microns and approximately 2.0 microns;

b) providing an inhalation delivery device; and,

c) delivering the composition to the respiratory tract by inhalation.

### BRIEF DESCRIPTION OF THE DRAWINGS

FIG. 1 is a graphical representation of the targeting and depot effect of liposomal amikacin showing microgram of antibacterial agent per gram of lung tissue against time for liposomal antibacterial agent delivered by inhalation and free antibacterial agent delivered by inhalation and IV.

FIG. 2 is a graphical representation of the biodistribution of ciprofloxacin in the lungs upon administration of ciprofloxacin in liposomal form by inhalation and in free form by inhalation and orally.

### DETAILED DESCRIPTION OF THE INVENTION

This invention is an inhalation system for the administration of antiinfective agents and the system's use in the treatment of diseases, particularly intracellular infections that involve uptake and transport of bacteria by the pulmonary macrophages of the lungs. The antiinfective agent are administered as a particle formulation. The particle formulations can comprise the antiinfective agent in particle form or a mixture of the antiinfective agent and one or more excipients, such as sugars, salts or complex carbohydrates. Sugars and other carbohydrates can be used as excipients and can include but are not limited to lactose, glucose, mannitol, dextrins, sucrose, maltose, halose, trehalose, and cyclodextrin The particle formulation of the ant linfective agent can comprise a non-covalent modification of the ant linfective agent, for example, a salt form of the ant linfective agent. The salt is preferably selected from the negative salt of the antiinfective agent. For example, the salt is selected from the sodium, potassium, lithium, sulfate, citrate or phosphate form of the ant linfective agent. More preferable salt forms of the antiinfective agent are a calcium, magnesium or iron salt of the antiinfective agent.

More preferably the particle formulation of the antiinfective agent can comprise a lipid or liposome formulation. The particle could comprise the antiinfective agent and one or more lipids, formulated as a lipid mixture. The optimal antiinfective agent to lipid ratio is from 10:1 to 1:1000 by weight. The lipid formulation could alternately be formulated as a lipid complex.

The lipids used in the formulation can be mixtures of phospholipids and/or steroids, such as cholesterol. Lipids used in the mixture can include phosphatidylcholines, steroids, phosphatidylglycerols, phosphatidylinositol, phosphatidylethanolamine, sphingomyelin, ceramides, glycolipids, and/or phosphatidylserines.

The lipid or liposome formulation can comprise the antiinfective agent and a mixture of phospholipids. Such a mixture could further comprise a mixture of one or more steroids.

In a most preferred embodiment the pharmaceutical formulation of the antiinfective agent could comprise a liposome, a lipid complex, a lipid clathrate or a proliposome.

The pharmaceutical formulation could alternately comprise a formulation of the antiinfective agent mixed with a polymer. The polymer could be: a polyester such as polyglycolic acid, polylactic acid, polycaprolactone, polydioxanone, trimetylene carbonate, polyester-polyethylene glycol copolymers, polyfumarates; poly amino acids such as poly ester-amides, tyrosine derived polycarbonates and polyacrylates, polyaspartates, polyglutamates, polyanhydrides, polyorthoesters, polyphazenes, polyurethanes, protein polymers, collagen, and polysaccharides such as chitin, hyaluronic acid, dextran and cellulosics. The association between polymer and antiinfective agent could be covalent, ionic, electrostatic, or steric.

Compositions are preferably adapted for use by inhalation, and more preferably for use in an inhalation delivery device for the composition's administration. The inhalation system can be used for the treatment of diseases in both man and animal, particularly lung disease.

The term “antiinfective agent” is used throughout the specification to describe a biologically active agent which can kill or inhibit the growth of certain other harmful or pathogenic organisms, including, but not limited to bacteria, yeast, viruses, protozoa or parasites and which can be administered to living organisms, especially animals such as mammals, particularly humans. The antiinfective agents includes but is not limited to antibacterial and antiviral agents. Antibacterial agents include, but are not limited to quinolones, such as ciprofloxicin, norfloxacin, ofloxacin, moxifloxacin, gatifloxacin, levofloxacin, lomefloxacin, sparfloxacin, cinoxacin, trovafloxacin, mesylate; tetracyclines particularly doxycycline and minocycline, oxytetracycline, demeclocycline, methacycline;isoniazid; penicillins, particularly penicillin g, penicillin v, penicillinase-resistant penicillins, isoxazolyl penicillins, amino penicillins, ureidopenicillins; cephalosporins; cephamycins such as cefoxitin, cefotetan, monobactams, aztreonam, loracarbef; carbapapenems such as imipenem, meropenem; β-lactamase inhibitors such as clavulanate, sulfactam, tazobactam; aminoglyclosides such as amikacin, streptomycin, gentamicin, tobramycin, netilmicin, kanamycin, macrolides such as erythromycin, rifampin, clarithromycin, azithromycin, dirithromycin, lincosamides such as lincomycin and clindamycin, glycopeptides such as vancomycin, teicoplanin, others chloramphenicol, trimethoprine/sulfamethoxazole, nitrofurantoin, oxazolidinone such as linezolid, streptogranin such as dalfopristin/quinupristin.

Antiviral agents include but are not limited to zidovudine, acyclovir, ganciclovir, vidarabine, idoxuridine, trifluridine, an interferon (e.g, interferon alpha-2a or interferon alpha-2b) and ribavirin.

Determination of compatibilities of the above listed agents and other ant infective agents with, and the amounts to be utilized in, compositions of the present invention are within the purview of the ordinarily skilled artisan to determine given the teachings of this invention. The physician can determine the amount of antiinfective agent to be administered based on the subject's age, condition, and the type and severity of infection. Generally the dose will be between 0.5 and 0.001 times the dose when the antiinfective agent is given orally or intravenously.

The term “intracellular infection” is used to describe infection where at least some of the infective agent resides inside a cell of the person or animal infected.

The lipids used in the compositions of the present invention can be synthetic, semi-synthetic or naturally-occurring lipids, including phospholipids, tocopherols, steroids, fatty acids, glycoproteins such as albumin, negatively-charged lipids and cationic lipids. Phosholipids include egg phosphatidylcholine (EPC), egg phosphatidylglycerol (EPG), egg phosphatidylinositol (EPI), egg phosphatidylserine (EPS), phosphatidylethanolamine (EPE), and egg phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid can be made up of fatty acids of different chain lengths and different degrees of unsaturation. In particular, the compositions of the formulations can include dipalmitoylphosphatidylcholine (DPPC), a major constituent of naturally-occurring lung surfactant as well as dioleoylphosphatidylcholine (DOPC). Other examples include dimyristoylphosphatidylcholine (DMPC) and dimyristoylphosphatidylglycerol (DMPG) dipalmitoylphosphatidcholine (DPPC) and dipalmitoylphosphatidylglycerol (DPPG) distearoylphosphatidylcholine (DSPC) and distearoylphosphatidylglycerol (DSPG), dioleylphosphatidylethanolamine (DOPE) and mixed phospholipids like palmitoylstearoylphosphatidylcholine (PSPC) and palmitoylstearoylphosphatidylglycerol (PSPG), and single acylated phospholipids like mono-oleoyl-phosphatidylethanolamine (MOPE).

The lipids used can include ammonium salts of fatty acids, phospholipids and glycerides, steroids, phosphatidylglycerols (PGs), phosphatidic acids (PAs), phosphotidylcholines (PCs), phosphatidylinositols (Pls) and the phosphatidylserines (PSs). The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2, 3-di-(9-(Z)-octadecenyloxy)-prop-1-yl-N,N,N-trimethylammonium chloride (DOTMA) and 1, 2-bis(oleoyloxy)-3-(trimethylammonio)propane (DOTAP). Examples of steroids include cholesterol and ergosterol. Examples of PGs, PAs, PIs, PCs and PSs include DMPG, DPPG, DSPG, DMPA, DPPA, DSPA, DMPI, DPPI, DSPI, DMPS, DPPS and DSPS, DSPC, DPPC, DMPC, DOPC, eggPC.

Liposomes composed of phosphatidylcholines, such as DPPC, aid in the uptake by the cells in the lung such as the alveolar macrophages and helps to sustain release of the antiinfective agent in the lung (Gonzales-Rothi et al. (1991)). The negatively charged lipids such as the PGs, PAs, PSs and PIs, in addition to reducing particle aggregation, can play a role in the sustained release characteristics of the inhalation formulation as well as in the transport of the formulation across the lung (transcytosis) for systemic uptake. The sterol compounds are believed to affect the release and leakage characteristics of the formulation.

The present invention covers the treatment of intracellular pulmonary infections that involve uptake and transport by the lung's macrophages in dissemination and persistence. These include but are not limited to, Bacillus anthracis, Listeria monocytogenes, Staphylococcus aureus,Salmenellolosis, Pseudomonas aeruginosa, Yersina pestis, Mycobacterium leprae, M. africanum, M. asiaticum, M. avium-intracellulare, M. chelonei subsp. abscessus, M. fallax, M. fortuitum, M. kansasii, M. leprae, M. malmoense, M. shimoidei, M. simiae, M. szulgai, M. xenopi, M. tuberculosis, Brucella melitensis, Brucella suis, Brucella abortus, Brucella canis, Legionella pneumonophilia, Francisella tularensis, Pneumocystis carinii, mycoplasma, including Mycoplasma penetrans and Mycoplasma pneumoniae, viral pneumonia, Hantavirus pulmonary syndrome, Respiratory syncytial virus, influenza.

Liposomes are completely closed lipid bilayer membranes containing an entrapped aqueous volume. Liposomes can be unilamellar vesicles (possessing a single membrane bilayer) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase. Lipid complexes are associations between lipid and the ant linfective agent that is being incorporated. This association can be covalent, ionic, electrostatic, noncovalent, or steric. These complexes are non-liposomal and are incapable of entrapping additional water soluble solutes. Examples of such complexes include lipid complexes of amphotericin B (Janoff et al. (1988) and cardiolipin complexed with doxorubicin.

A lipid clathrate is a three-dimensional, cage-like structure employing one or more lipids wherein the structure entraps a bioactive agent. Such clathrates, when a component of a particle, are included within the scope of the present invention.

Proliposomes are formulations that can become liposomes or lipid complexes upon coming in contact with an aqueous liquid. Agitation or other mixing can be necessary. Such proliposomes, when a component of a particle, are included within the scope of the present invention.

Liposomes can be produced by a variety of methods (for example, see, Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization can be used to produce smaller unilamellar liposomes from larger liposomes (see, for example, Paphadjopoulos et al. (1968); Deamer and Uster (1983); and Chapman et al. (1968)).

The original liposome preparation of Bangham et al. (J. Mol. Biol., 1965, 13:238-252) involves suspending phospholipids in an organic solvent which is then evaporated to dryness leaving a phospholipid film on the reaction vessel. Next, an appropriate amount of aqueous phase is added, the mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes can be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467), the pertinent portions of which are also incorporated herein by reference.

Other techniques that are used to prepare vesicles include those that form reverse-phase evaporation vesicles (REV), Papahadjopoulos et al., U.S. Pat. No. 4,235,871. Another class of liposomes that can be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

A variety of sterols and their water soluble derivatives such as cholesterol hemisuccinate have been used to form liposomes; see specifically Janoff et al., U.S. Pat. No. 4,721,612, issued Jan. 26, 1988, entitled “Steroidal Liposomes.” Mayhew et al, described a method for reducing the toxicity of antibacterial agents and antiviral agents by encapsulating them in liposomes comprising alpha-tocopherol and certain derivatives thereof. Also, a variety of tocopherols and their water soluble derivatives have been used to form liposomes, see Janoff et al., U.S. Pat. No. 5,041,278.

A process for forming liposomes or lipid complexes involves the infusion of lipids dissolved in ethanol into an aqueous phase containing the antiinfective agent. This is done below the bilayer phase transition of the highest melting lipid. The ethanol/aqueous phase ratio is approximately 1:2. The ethanol and unentrapped antiinfective agent can be removed by a washing step such as centrifugation, dialysis, or diafiltration. The washing step is also performed below the bilayer phase transition of the highest melting lipid.

It is of importance to note that any of the above described methods of forming liposomes can, depending on the lipid composition and antiinfective agent properties, result in the formation of a lipid complex, not a liposome.

In a liposome-antiinfective agent delivery system, an antiinfective agent is entrapped in the liposome and then administered to the patient to be treated. For example, see Rahman et al., U.S. Pat. No. 3,993,754; Sears, U.S. Pat. No. 4,145,410; Paphadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578. Alternatively, if the bioactive agent is lipophilic, it may associate with the lipid bilayer. In the present invention, the term “entrapment” shall be taken to include both the antiinfective agent in the aqueous volume of the liposome as well as antiinfective agent associated with the lipid bilayer. The bioactive agent can also be associated or complexed with a liposome through a covalent, electrostatic, hydrogen bonded or other association.

The term “particle size” refers to the diameter of the particle, liposome or lipid complex, or, in the case of a non-spherical particle, liposome or lipid complex, the largest dimension. Particle size can be measured by a number of techniques well known to ordinarily skilled artisans, such as quasi-electric light scattering. In the present invention the particles generally have a diameter of between about 0.01 microns and about 6.0 microns, preferably between approximately 0.01 microns and approximately 2.0 microns, more preferably between approximately 0.01 microns and approximately 1.0 microns. Even more preferably the particle diameter is between approximately 0.01 microns and approximately 0.5 microns.

Liposome or lipid complex sizing can be accomplished by a number of methods, such as extrusion, sonication and homogenization techniques which are well known, and readily practiced, by ordinarily skilled artisans. Extrusion involves passing liposomes, under pressure, one or more times through filters having defined pore sizes. The filters are generally made of polycarbonate, but the filters may be made of any durable material which does not interact with the liposomes and which is sufficiently strong to allow extrusion under sufficient pressure. Preferred filters include “straight through” filters because they generally can withstand the higher pressure of the preferred extrusion processes of the present invention. “Tortuous path” filters may also be used. Extrusion can also use asymmetric filters, such as AnotecO™ filters, which involves extruding liposomes through a branched-pore type aluminum oxide porous filter.

Liposomes or lipid complexes can also be size reduced by sonication, which employs sonic energy to disrupt or shear liposomes, which will spontaneously reform into smaller liposomes. Sonication is conducted by immersing a glass tube containing the liposome suspension into the sonic epicenter produced in a bath-type sonicator. Alternatively, a probe type sonicator may be used in which the sonic energy is generated by vibration of a titanium probe in direct contact with the liposome suspension. Homogenization and milling apparatii, such as the Gifford Wood homogenizer, Polytron™ or Microfluidizer™, can also be used to break down larger liposomes or lipid complexes into smaller liposomes or lipid complexes.

The resulting liposomes can be separated into homogeneous populations using methods well known in the art; such as tangential flow filtration. In this procedure, a heterogeneously sized population of liposomes or lipid complexes is passed through tangential flow filters, thereby resulting in a liposome population with an upper and/or lower size limit. When two filters of differing sizes, that is, having different pore diameters, are employed, liposomes smaller than the first pore diameter pass through the filter. This filtrate can the be subject to tangential flow filtration through a second filter, having a smaller pore size than the first filter. The retentate of this filter is a liposome population having upper and lower size limits defined by the pore sizes of the first and second filters, respectively.

Mayer et al. found that the problems associated with efficient liposomal entrapment of lipophilic ionizable bioactive agents such as antineoplastic agents, for example, anthracyclines or vinca alkaloids, can be alleviated by employing transmembrane ion gradients. Aside from inducing greater uptake, such transmembrane gradients also act to increase antiinfective agent retention in the liposomes.

Liposomes or lipid complexes themselves have been reported to have no significant toxicities in previous human clinical trials where they have been given intravenously (Richardson et al., (1979), Br. J. Cancer 40:35; Ryman et al., (1983) in “Targeting of Antiinfective agents” G. Gregoriadis, et al., eds. pp 235-248, Plenum, N.Y.; Gregoriadis G., (1981), Lancet 2:241, and Lopez-Berestein et al., (1985)). Liposomes are reported to concentrate predominately in the reticuloendothelial organs lined by sinosoidal capillaries, i.e., liver, spleen, and bone marrow, and phagocytosed by the phagocytic cells present in these organs.

The therapeutic properties of many ant linfective agents can be dramatically improved by the intravenous administration of the agent in a liposomally encapsulated form (See, for example, Shek and Barber (1986)). Toxicity can be reduced, in comparison to the free form of the antiinfective agent, meaning that a higher dose of the liposomally encapsulated antiinfective agent can safely be administered (see, for example, Lopez-Berestein, et al. (1985) J. Infect. Dis., 151:704; and Rahman, et al. (1980) Cancer Res., 40:1532). Benefits obtained from liposomal encapsulation likely result from the altered pharmacokinetics and biodistribution of the entrapped ant linfective agent. A number of methods are presently available for “charging” liposomes with bioactive agents (see, for example, Rahman et al., U.S. Pat. No. 3,993,754; Sears, U.S. Pat. No. 4,145,410; Papahadjopoulos, et al., U.S. Pat. No. 4,235,871; Lenk et al., U.S. Pat. No. 4,522,803; and Fountain et al., U.S. Pat. No. 4,588,578). Ionizable bioactive agents have been shown to accumulate in liposomes in response to an imposed proton or ionic gradient (see, Bally et al., U.S. Pat. No. 5,077,056; Mayer, et al. (1986); Mayer, et al. (1988); and Bally, et al. (1988)). Liposomal encapsulation could potentially provide numerous beneficial effects for a wide variety of bioactive agents and a high bioactive agent to lipid ratio should prove important in realizing the potential of liposomally encapsulated agents.

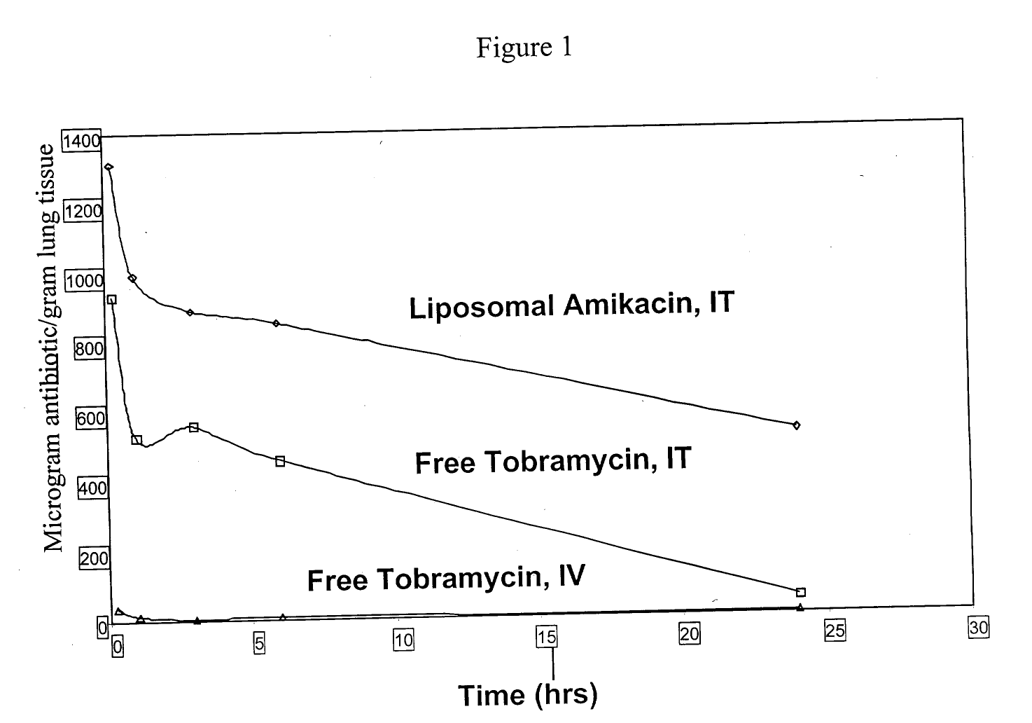

As can be seen in FIG. 1 which compares the micrograms of antibacterial agent per gram of lung tissue, a much larger deposition of aminoglycoside can be delivered intratracheally compared to injection. Without being bound to a particular theory, it appears that the depot effect is also demonstrated, in that greater than a ten-fold increase in antibacterial agent remains following twenty four hours. Thus, the therapeutic level of antibacterial agent is maintained for a longer period of time in the liposomal formulations of amikacin compared to free tobramycin.

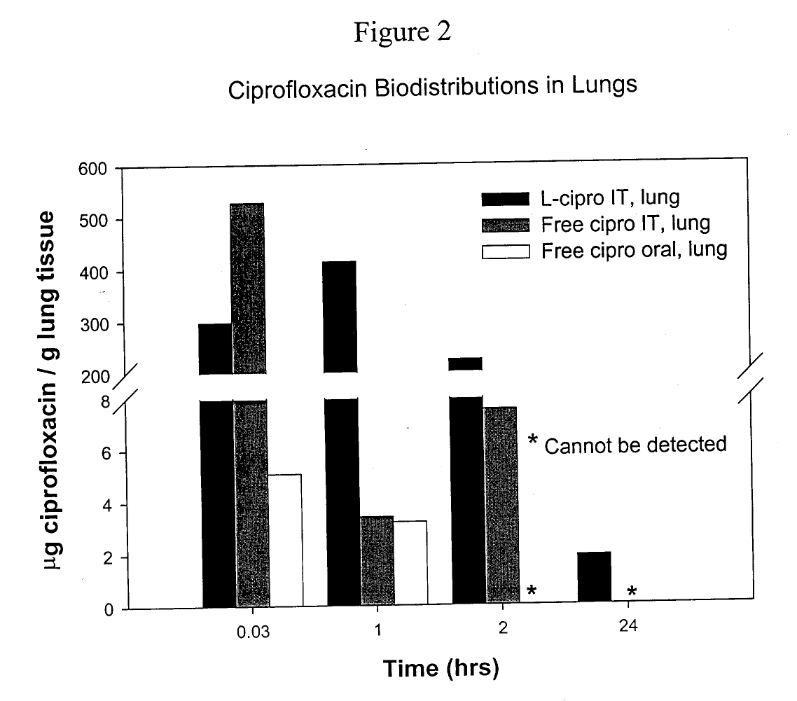

As shown in FIG. 2, liposomal ciprofloxicin administered intratracheally is maintained at a high level in the lungs for two hours whereas the lung levels of free ciprofloxicin delivered intratracheally were negligible after one hour. For orally delivered ciprofloxicin the lung concentration was one hundredth the concentration of liposomal ciprofloxicin administered by intratracheal administration. Only liposomal ciprofloxicin delivered intratracheally was detectable in the lungs after 24 hours. Thus liposomal ciprofloxicin given by inhalation is more advantageous with respect to targeting and retention in the lung than free ciprofloxicin given either by inhalation or orally.

The inhalator can be an aerosolizer, a nebulizer or a powder-administering device. It can deliver multiple doses or a single dose. A metered dose inhaler (MDI) can be used or a dry power inhaler can be employed as the inhalator. Ultrasonic, electrical, pneumatic, hydrostatic or mechanical forces such as (compressed air, or by other gases) can drive the device. The inhalation antiinfective agent delivery system can resuspend particles, or generate aerosol particles.

The inhalator can be a nebulizer, which will deliver fine mists of either liquids, suspensions or dispersions for inhalation. The devices can be mechanical powder devices which disperse fine powder into a finer mist using leverage or piezo electric charges in combination with suitably manufactured porous filter discs, or as formulations that do not aggregate in the dose chamber. Propellants can be used to spray a fine mist of the product such as fluorochlorocarbons, fluorocarbons, nitrogen, carbon dioxide, or other compressed gases.

A nebulizer type inhalation delivery device can contain the compositions of the present invention as a solution, usually aqueous, or a suspension. In generating the nebulized spray of the compositions for inhalation, the nebulizer type delivery device can be driven ultrasonically, by compressed air, by other gases, electronically or mechanically. The ultrasonic nebulizer device generally works by imposing a rapidly oscillating waveform onto the liquid film of the formulation via an electrochemical vibrating surface. At a given amplitude the waveform becomes unstable, disintegrates the liquids film, and produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer can be portable and hand held in design, and can be equipped with a self contained electrical unit. The nebulizer device can consist of a nozzle that has two coincident outlet channels of defined aperture size through which the liquid formulation can be accelerated. This results in impaction of the two streams and atomization of the formulation. The nebulizer can use a mechanical actuator to force the liquid formulation through a multiorifice nozzle of defined aperture size(s) to produce an aerosol of the formulation for inhalation. In the design of single dose nebulizers, blister packs containing single doses of the formulation can be employed. The nebulizer can also be used to form the desired liposomes or lipid complexes.

A metered dose inhalator (MDI) can be employed as the inhalation delivery device of the inhalation system. This device is pressurized and its basic structure consists of a metering valve, an actuator and a container. A propellant is used to discharge the formulation from the device. The composition can consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. Traditional chloroflourocarbons like CFC-1 1, 12 and 114 are used only when essential. The device of the inhalation system can deliver a single dose via, e.g., a blister pack, or it can be multi dose in design. The pressurized metered dose inhalator of the inhalation system can be breath actuated to deliver an accurate dose of the lipid based formulation. To insure accuracy of dosing, the delivery of the formulation can be programmed via a microprocessor to occur at a certain point in the inhalation cycle. The MDI can be portable and hand held.

A dry powder inhalator (DPI) can be used as the inhalation delivery device of the inhalation system. This device's basic design consists of a metering system, a powdered composition and a method to disperse the composition. Forces like rotation and vibration can be used to disperse the composition. The metering and dispersion systems can be mechanically or electrically driven and can be microprocessor programmable. The device can be portable and hand held. The inhalator can be multi or single dose in design and use such options as hard gelatin capsules, and blister packages for accurate unit doses. The composition can be dispersed from the device by passive inhalation; i.e., the patient's own inspiratory effort, or an active dispersion system can be employed. The dry powder of the composition can be sized via processes such as jet milling, spray dying and supercritical fluid manufacture. Acceptable excipients such as the sugars mannitol and maltose can be used in the preparation of the powdered formulations. These are particularly important in the preparation of freeze dried liposomes and lipid complexes. These sugars help in maintaining the liposome's physical characteristics during freeze drying and minimizing their aggregation when they are administered by inhalation. The sugar by its hydroxyl groups can help the vesicles maintain their tertiary hydrated state and help minimize particle aggregation.

The antiinfective agent formulation of the inhalation system can contain more than one antiinfective agent (e.g., two antiinfective agents for a synergistic effect).

In addition to the above discussed lipids and albumin and antiinfective agent(s), the composition of the antiinfective agent formulation of the inhalation system can contain excipients (including solvents, salts and buffers), preservatives and surfactants that are acceptable for administration by inhalation to humans or animals.

The term “treatment” or “treating” means administering a composition to an animal such as a mammal or human for preventing, ameliorating, treating or improving a medical condition.

The term “infective agent” refers to a harmful or pathogenic organism, including, but not limited to, bacteria, yeast, viruses, protozoa or parasites.

The term “pharmaceutical formulation comprising a particle” refers to a formulation of the antiinfective agent where the antiinfective agent is present in a particle form. Without limiting the claims, “particle” refers to a primarily pure particle, a particle of antiinfective agent mixed with one or more excipients, a covalent modification of the antiinfective agent, a particle wherein the antiinfective agent is mixed with lipids, a particle wherein the antiinfective agent is mixed with phospholipids, a particle wherein the antiinfective agent is formulated as part of a lipid complex such as a liposome, a particle wherein the antiinfective agent is present in association with a liposome, a particle wherein the antiinfective agent is present in association with a lipid clathrate or a particle wherein the antiinfective agent is present as a polymer formulation. In the case of inhalation by nebulization the term “particle” does not refer to the droplet which is released from the nebulizer but only to the antiinfective agent particle contained within or associated with the droplet.

In general, the doses of the antiinfective agent will be chosen by a physician based on the age, physical condition, weight and other factors known in the medical arts.

### EXAMPLE 1

0.734 g DPPC, 0.232 g CHOL, 0.079 g DOPC, and 0.096 g DOPG were dissolved in 35.3 mL of EtOH, which equals 32.2 mg total lipid/1 mL EtOH. 8.6 g of Amikacin Sulfate (“Amk”) was dissolved in 114.7 mL of buffer (10 mM Hepes 150 mM NaCl @ pH 6.8). Amk concentration in the buffer was 74.9 mg/mL. The solution became acidic so the pH of the antiinfective agent/buffer solution was adjusted using NaOH to give desired pH 6.8. With a filtered syringe, the EtOH/lipid was slowly added to the Amk/buffer to give a total sample volume of 150 mL. The sample was allowed to sit for half and hour before dialysis. The pharmacokinetics of aminoglycoside was determined in rats following intratracheal (IT) administration of either free tobramycin, Chiron or liposomal amikacin. This was compared to the distribution obtained in the lungs following a tail vein injection of free tobramycin. In all cases a dose of 4 mg/kg was administered. As can be seen in FIG. 1 by comparing the micrograms of antibacterial agent per gram of lung tissue, a much larger deposition of aminoglycoside can be delivered by IT compared to injection. Without being bound to a particular theory, it appears that the depot effect is also demonstrated, in that greater than a ten-fold increase in antibacterial agent remains following twenty four hours. Thus, the therapeutic level of antibacterial agent is maintained for a longer period of time in the liposomal formulations of amikacin compared to free tobramycin.

### EXAMPLE 2

141.7 mg DPPC and 8.3 mg cholesterol were dissolved in chloroform, then rotoevaporated and left overnight on a vacuum to remove the chloroform. The resulting thin film was then hydrated with 1.5 mL of citrate buffer at pH 5 to give a 100 mg/ml multi-lamellar vesicle (MLV) solution. The MLV solution was then sonicated until small unilamellar vesicles (SUVs) were formed (1 hour). A 16 mg/ml stock Cipro solution in citrate buffer at pH 5 was prepared. These were mixed as follows.

0.764 mL SUV(100 mg/ml) was added to 0.764(16 mg/ml Cipro Stock) and 0.470 mL EtOH to produce a 2 mL sample volume. The sample was then dialyzed in citrate buffer at pH 5.

The pharmacokinetics of ciprofloxicin was determined in mice following intratracheal (IT) administration of either free ciprofloxicin or liposomal ciprofloxicin. The distribution following IT administration was compared with the distribution obtained in the lungs following an oral delivery of ciprofloxicin. As shown in FIG. 2, liposomal ciprofloxicin administered IT is maintained at a high level in the lungs for two hours whereas the lung levels of free ciprofloxicin delivered IT was negligible after one hour. For orally delivered ciprofloxicin the lung concentration was one hundredth the concentration of liposomal ciprofloxicin administered by IT administration. Only liposomal ciprofloxicin delivered by IT administration was detectable in the lungs after 24 hours.

What is claimed:

1. A system for delivery of an antiinfective agent comprising:
a) a pharmaceutical formulation comprising a particle comprising an antiinfective agent directed to prevention and treatment of intracellular infections in the lung caused by an infective agent, the pharmaceutical formulation comprising particles with a diameter of between approximately 0.01 microns and approximately 2.0 microns and,
b) an inhalation delivery device.

2. The system of claim 1 wherein the particles have a diameter of between approximately 0.01 microns and approximately 1.0 micron.

3. The system of claim 1 wherein the particles have a diameter of between approximately 0.01 microns and approximately 0.5 microns.

4. The system of claim 1 wherein the particles have a diameter of between approximately 0.02 microns and approximately 0.5 microns.

5. The system of claim 1, wherein the infective agent is a bacteria.

6. The system of claim 5, wherein the bacteria is selected from Bacillus anthracis, Listeria monocytogenes, Staphylococcus aureus, Salmenellolosis, Pseudomonas aeruginosa, Yersina pestis, Mycobacterium leprae, M. africanum, M. asiaticum, M. avium-intracellulare, M. chelonei subsp. abscessus, M. fallax, M. fortuitum, M. kansasii, M. leprae, M. malmoense, M. shimoidei, M. simiae, M. szulgai, M. xenopi, M. tuberculosis, Brucella melitensis, Brucella suis, Brucella abortus, Brucella canis, Legionella pneumonophilia, Francisella tularensi, pneumocystis carinii and mycoplasma.

7. The system of claim 6 wherein the bacteria is Bacillus anthracis.

8. The system of claim 6 wherein the bacteria is Mycobacterium leprae.

9. The system of claim 6 wherein the bacteria is M.tuberculosisI.

10. The system of claim 1, wherein the infective agent is a virus.

11. The system of claim 1, wherein the virus is selected from hantavirus, respiratory syncytial virus, influenza, and viral pneumonia.

12. The system of claim 1, wherein the pharmaceutical formulation comprises the antiinfective agent in particle form.

13. The system of claim 1, wherein the pharmaceutical formulation comprises a mixture of the antiinfective agent and one or more excipients.

14. The system of claim 13, wherein the one or more excipients are selected from sugars, salts and polymers.

15. The system of claim 1, wherein the pharmaceutical formulation comprises a non-covalent modification of the antiinfective agent.

16. The system of claim 7, wherein the non-covalent modification of the antiinfective agent is a salt.

17. The system of claim 8, wherein the salt is selected from the sodium, potassium, lithium, sulfate, citrate, phosphate, calcium, magnesium or iron salt of the antiinfective agent.

18. The system of claim 1, wherein the pharmaceutical formulation comprises the ant linfective agent and one or more lipids, the antiinfective agent and the one or more lipids being formulated as a lipid mixture.

19. The system of claim 10, wherein the antiinfective agent to lipid ratio is from 10:1 to 1:1000 by weight.

20. The system of claim 1, wherein the pharmaceutical formulation comprises the antiinfective agent and a mixture of phospholipids.

21. The system of claim 12, wherein the mixture of phospholipids comprises one or more phospholipids selected from the group consisting of phosphatidylcholines, phosphatidylglycerols, phosphatidylserines, phosphotidylinositols, phosphatidylethanolamines, sphingomyelins, ceramides, and steroids.

22. The system of claim 12, wherein the pharmaceutical formulation further comprises a mixture of one or more steroids.

23. The system of claim 1, wherein the pharmaceutical formulation comprises the antiinfective agent and a lipid, the antiinfective agent and the lipid being formulated as a lipid complex.

24. The system of claim 1, wherein the pharmaceutical formulation comprises a liposome.

25. The system of claim 16, wherein the liposome is a multilamellar vesicle.

26. The system of claim 16, wherein the liposome is a small unilamellar vesicle.

27. The system of claim 1, wherein the pharmaceutical formulation comprises a lipid complex with a diameter of from approximately 0.01 microns to approximately 6.0 microns.

28. The system of claim 27, wherein the pharmaceutical formulation comprises a lipid complex with a diameter of from approximately 0.01 microns to approximately 0.5 microns.

29. The system of claim 1, wherein the pharmaceutical formulation comprises a lipid clathrate with a diameter of from approximately 0.01 microns to approximately 6.0 microns.

30. The system of claim 29, wherein the pharmaceutical formulation comprises a lipid clathrate with a diameter of from approximately 0.01 microns to approximately 0.5 microns.

31. The system of claim 1, wherein the pharmaceutical formulation comprises a proliposome.

32. The system of claim 1, wherein the pharmaceutical formulation comprises a polymer formulation of the antiinfective agent.

33. The system of claim 1, wherein the antiinfective agent directed to treatment of intracellular infection is a quinolone.

34. The system of claim 33 wherein the quinolone is ciprofloxicin, norfloxacin, ofloxacin, moxifloxacin or levofloxacin.

35. The system of claim 34 wherein the quinolone is ciprofloxacin.

36. The system of claim 1, wherein the antiinfective agent directed to treatment of intracellular infection is a tetracycline.

37. The system of claim 36 wherein the tetracycline is doxycycline, minocycline, oxytetracycline, demeclocycline, or methacycline.

38. The system of claim 1, wherein the antiinfective agent directed to treatment of intracellular infection is a penicillin.

39. The system of claim 38 wherein the antiinfective agent additionally comprises a beta lactamase inhibitor.

40. The system of claim 38 wherein the penicillin is penicillin G, penicillin V, a penicillinase-resistant penicillin, an isoxazolyl penicillin, an amino penicillin, or a ureidopenicillin.

41. The system of claim 1, wherein the antiinfective agent directed to treatment of intracellular infection is a cephalosporin.

42. The system of claim 41, wherein the antiinfective agent additionally includes a beta lactamase inhibitor.

43. The system of claim 42 wherein the beta lactamase inhibitor is clavulanate, sulfactam, or tazobactam.

44. The system of claim 1, wherein the antiinfective agent directed to treatment of intracellular infection is a macrolide.

45. The system of claim 44, wherein the macrolide is erythromycin, rifampin, clarithromycin, dirithromycin or troleandomycin

46. The system of claim 1, wherein the antiinfective agent directed to treatment of intracellular infection is an aminoglycoside.

47. The system of claim 46, wherein the aminoglycoside is amikacin, streptomycin, gentamycin, tobramycin, netilmicin, or kanamycin.

48. The system of claim 47, wherein the aminoglycoside is amikacin.

49. The system of claim 47, wherein the aminoglycoside is tobramycin.

50. The system of claim 47 wherein the aminoglycoside is gentamycin.

51. The system of claim 1, wherein the antiinfective agent directed to treatment of intracellular infection is a glycopeptide.

52. The system of claim 51, wherein the glycopeptide is vancomycin or teicoplanin.

53. The system of claim 1, wherein the antiinfective agent directed to treatment of intracellular infection is a cephamycin.

54. The system of claim 53, wherein the cephamycin is cefoxitin or cefotetan.

55. The system of claim 1, wherein the antiinfective agent directed to treatment of intracellular infection is a monobactam.

56. The system of claim 55 wherein the monobactam is aztreonam.

57. The system of claim 1, wherein the antiinfective agent directed to treatment of intracellular infection is a carbapapenem.

58. The system of claim 57 wherein the carbapapenem is imipenem or meropenem.

59. The system of claim 1, wherein the antiinfective agent directed to treatment of intracellular infection is a lincosamide.

60. The system of claim 59 wherein the lincosamide is lincomycin or clindamycin.

61. The system of claim 1, wherein the ant linfective agent directed to treatment of intracellular infection is an oxazolidinone.

62. The system of claim 61 wherein the oxazolidinone is linezolid.

63. The system of claim 1, wherein the antiinfective agent directed to treatment of intracellular infection is a streptogranin.

64. The system of claim 63 wherein the streptogranin is dalfopristin or quinupristin.

65. The system of claim 1, wherein the antiinfective agent directed to treatment of intracellular infection is chloramphenicol.

66. The system of claim 1, wherein the antiinfective agent directed to treatment of intracellular infection is trimethoprine.

67. The system of claim 1, wherein the antiinfective agent directed to treatment of intracellular infection is sulfamethoxazole.

68. The system of claim 1, wherein the antiinfective agent directed to treatment of intracellular infection is nitrofurantoin.

69. The system of claim 1, wherein the inhalation delivery device is an aerosolizer.

70. The system of claim 1, wherein the inhalation delivery device is a nebulizer.

71. The system of claim 1, wherein the inhalation delivery device is a powder administering device.

72. The system of claim 1, wherein the intracellular infection is Bacillus antracis and the antiinfective agent is ciprofloxacin.

73. The system of claim 1, wherein the intracellular infection is M.tuberculosis and the antiinfective agent is isoniazid.

74. A method for treatment of intracellular infection comprising:
a) providing a pharmaceutical formulation of a particle comprising an antiinfective agent, the antiinfective agent being directed to treatment of intracellular infections in the lung, the pharmaceutical formulation comprising particles with a diameter of between approximately 0.01 microns and approximately 2.0 microns;
b) providing an inhalation delivery device; and,
c) delivering the composition to the respiratory tract by inhalation.

75. The method of claim 74, wherein the particles have a diameter of between approximately 0.01 microns and approximately 1.0 microns.

76. The method of claim 74, wherein the particles have a diameter of between approximately 0.01 microns and approximately 0.5 microns.

77. The method of claim 74, wherein the infective agent is a bacteria.

78. The method of claim 77 wherein the bacteria is selected from Bacillus anthracis, Listeria monocytogenes, Staphylococcus aureus, Salmenellolosis, Pseudomonas aeruginosa, Yersina pestis, Mycobacterium leprae, M. africanum, M. asiaticum, M. avium-intracellulare, M. chelonei subsp. abscessus, M. fallax, M. fortuitum, M. kansasii, M. leprae, M. malmoense, M. shimoidei, M. simiae, M. szulgai, M. xenopi, M. tuberculosis, Brucella melitensis, Brucella suis, Brucella abortus, Brucella canis, Legionella pneumonophilia, Francisella tularensis, pneumocystis carinii or mycoplasma.

79. The method of claim 78 wherein the bacteria is Bacillus anthracis.

80. The method of claim 78 wherein the bacteria is Mycobacterium leprae.

81. The method of claim 78 wherein the bacteria is M. tuberculosis.

82. The method of claim 78 wherein the bacteria is Legionella pneumonophilia.

83. The method of claim 74 wherein the infective agent is a virus.

84. The method of claim 83 wherein the virus is selected from from hantavirus, respiratory syncytial virus, influenza, and viral pneumonia.

85. The method of claim 74 wherein the antiinfective agent is in particle form.

86. The method of claim 74 wherein the inhalation delivery device comprises an aerosolizer.

87. The method of claim 74 wherein the inhalation delivery device comprises a nebulizer.

88. The method of claim 74 wherein the inhalation delivery device comprises a dry powder inhalator.

89. The method of claim 74 wherein the antiinfective agent is formulated as a lipid mixture.

90. The method of claim 74 wherein the ant linfective agent is formulated as a lipid complex.

91. The method of claim 74 wherein the antiinfective agent is incorporated into a liposome.

92. The method of claim 74, wherein the pharmaceutical formulation comprises a lipid complex with a diameter of from approximately 0.01 microns to approximately 0.5 microns.

93. The method of claim 74, wherein the pharmaceutical formulation comprises a lipid clathrate with a diameter of from approximately 0.01 microns to approximately 0.5 microns.

94. The method of claim 74, wherein the pharmaceutical formulation comprises a proliposome.
